(19) United States
(12) Patent Application Publication
(10) Pub. No.: US 2013/0137971 A1
(43) Pub. Date: May 30, 2013

(54) MULTIPLE-PERSON MRI SCANNER WITH TWIN HEAD COIL

(71) Applicant: Ray F. Lee, Princeton, NJ (US)

(72) Inventor: Ray F. Lee, Princeton, NJ (US)

(21) Appl. No.: 13/749,207

(22) Filed: Jan. 24, 2013

Related U.S. Application Data
(62) Division of application No. 12/608,978, filed on Oct. 29, 2009.

(51) Int. Cl. A61B 5/055

(52) U.S. Cl. 600/422

(57) ABSTRACT

A system and method perform magnetic resonance imaging (“MRI”) on more than one people simultaneously with one MRI scanner. The system may include a superconducting or non-superconducting magnet, a set of three dimensions of magnetic field gradients, RF coils, and magnetic field shimming coil set that can accommodate more than one people within one scanner. The system and method may be used for performing MRI on more than one interacting or non-interacting people for anatomic, functional, metabolic, and/or molecular imaging and studies.

This application is a divisional of U.S. patent application Ser. No. 12/608,978, filed Oct. 29, 2009, which is incorporated by reference as if fully set forth.

### FIELD OF THE INVENTION

The present invention relates generally to Magnetic Resonance Imaging (“MRI”). More specifically, the present invention relates to systems and methods for performing MRI on more than one interacting or non-interacting people simultaneously within one MRI scanner for anatomic, functional, metabolic, and/or molecular imaging and studies.

### BACKGROUND INFORMATION

One of the major functions of the human brain is to mediate interactions with other people, such as inter-personal communication and physical contact. Understanding such dynamic interactions between two minds is essential for characterizing human social behavior.

Until recently, studying brain mechanisms underlying social interactions has not been possible due to the lack of measurable methods to observe two interacting minds simultaneously. However, some progress has been made during the past ten years [1]. For example, Montague and colleagues developed a technique known as “hyperscanning” that uses the internet to connect and synchronize two MRI scanners while subjects were scanned performing a simple deception game [2]. Similarly, Babiloni and colleagues studied dyads participating in the Prisoner's Dilemma paradigm while recording EEG signals [3].

In line with this research, we have developed a novel MRI head coil that allows for the acquisition of fMRI signals from at least two subjects' brains while the subjects are in close proximity to one another, allowing for social interactions not possible with the remote hyperscanning technique. Unlike hyperscanning, which scans two people in separate scanners with only a visual or audio connection between them, the proposed device allows for fMRI studies of two people within close physical contact.

### SUMMARY OF THE INVENTION

In view of the foregoing, one of the objects of the present invention is to provide systems and methods for performing MR on more than one people simultaneously within one MRI scanner.

It is another object of the present invention to provide systems and methods for design and manufacture a MRI scanner which includes at least a superconducting or non-superconducting magnet, a set of three dimensions of magnetic field gradients, RF coils, and magnetic field shimming coil set that can accommodate more than one people within one scanner.

It is another object of the present invention to provide systems and methods for performing MRI on more than one interacting or non-interacting people for anatomic, functional, metabolic, and/or molecular imaging and studies.

These and other objects of the present invention are accomplished using an exemplary embodiment which can include a twin-head coil comprising: two circular polarized volume coils side-by-side for two people; the two component coils are decoupled by both their geometrical orientation and decoupling interface circuit system [4].

### BRIEF DESCRIPTION OF THE DRAWINGS

The foregoing and other objects of the present invention will be apparent upon consideration of the following detailed description, taken in conjunction with the accompanying drawings, in which like reference characters refer to like parts throughout, and in which:

FIG. 1 is a visual illustration of an exemplary embodiment of an elliptical superconducting magnet according to the present invention;

FIG. 2 is a visual picture of an exemplary embodiment of the twin-head coil according to the present invention;

FIG. 3 is a visual diagram of exemplary layouts of the interface between the twin-head coil and MRI scanner in accordance with an exemplary embodiment of the present invention.

### DETAILED DESCRIPTION OF THE INVENTION

An apparatus for simultaneously acquiring magnetic resonance signals from more than one person in one magnetic resonance imaging scanner, may include: a) an arbitrary shape of inner bore superconducting or non-superconducting magnet that can generate homogeneous static magnetic field whose volume can accommodate more than one person; b) three direction magnetic field gradient coils which can produce linear magnetic gradient region that can accommodate more than one people; c) RF coils that can transmit and/or receive MR signals in the region that can accommodate more than one person; and d) a shimming coil set that can shim an elliptical volume that is large enough for accommodating more than one people.

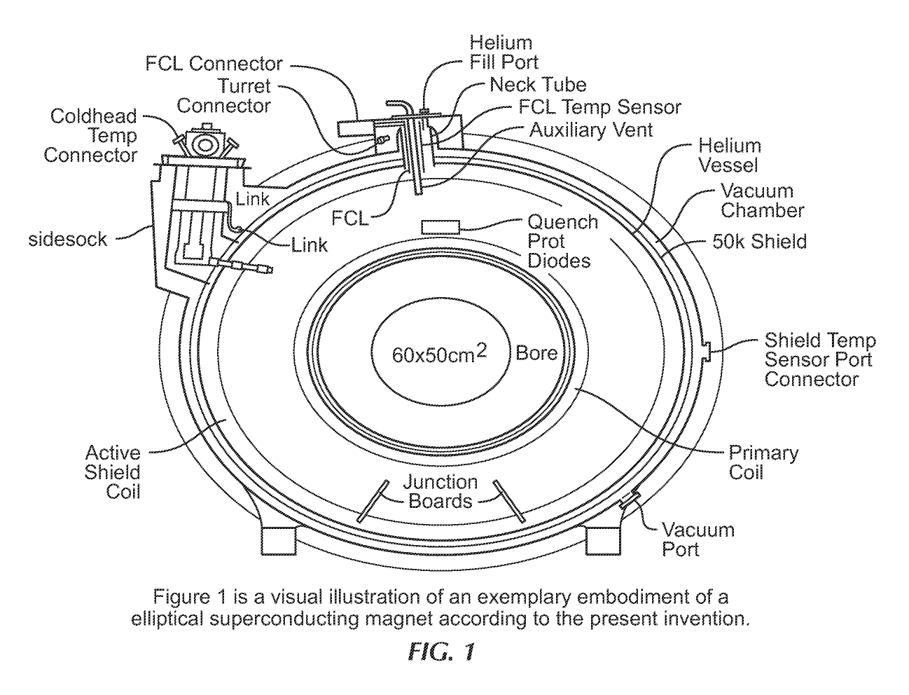

FIG. 1 is a diagram of an elliptical superconducting magnet. The elliptical superconducting magnet includes an elliptical inner bore surrounded by a primary coil. The elliptical superconducting magnet also includes an active shield coil, vacuum port, shield temp sensor port connector, 50k shield, vacuum chamber, quench prot diodes, junction boards, helium vessel, helium fill port, neck tube, FCL, FCL connector, FCL temp sensor, Auxiliary vent, turret connection, coolhead temp connector and sidesock.

The apparatus may include: a) an elliptical cylinder inner bore superconducting or non-superconducting magnet that can generate homogeneous static magnetic field whose volume can accommodate two people, as shown in FIG. 1; b) Three direction magnetic field gradient coils which can produce linear magnetic gradient region that can accommodate two people; c) RF coils that can transmit and/or receive MR signals in the region that can accommodate two people; and d) a shimming coil set that can shim an elliptical volume that is large enough for accommodating two people.

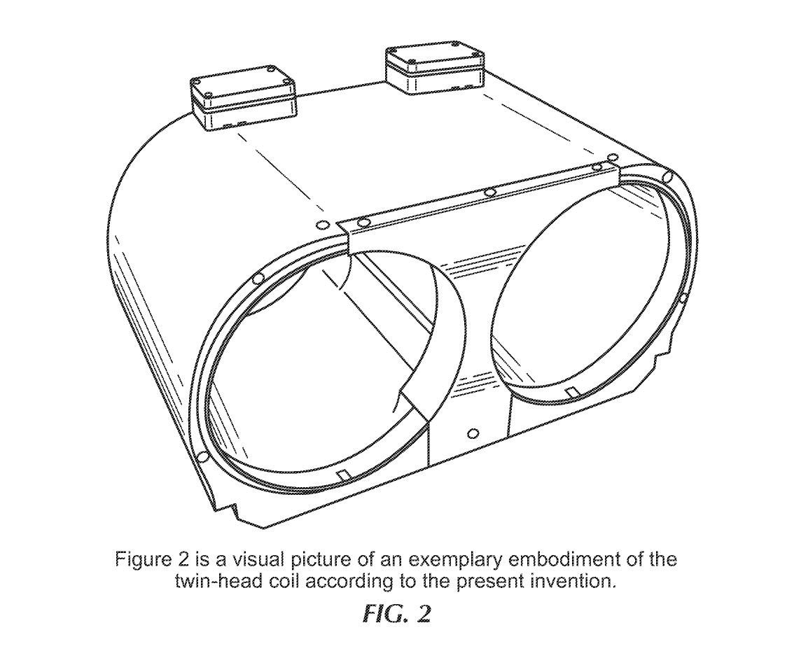

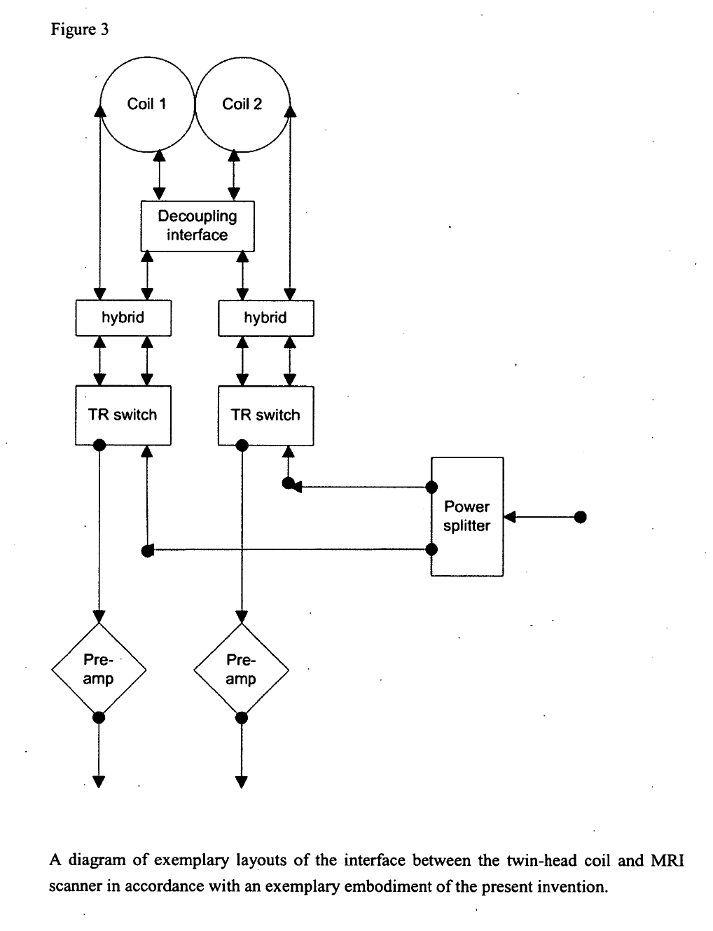

The apparatus may further include a twin-head coil comprising: a) two circular polarized volume coils side-by-side for two people, as shown in FIG. 2; b) The two component coils are decoupled by both their geometrical orientation and a decoupling interface circuit system; and c) the interface between MRI scanner and the twin-head coil is shown in FIG. 3. Component b) may further include a scheme for decoupling circular-polarized RF coils.

A method for simultaneously acquiring magnetic resonance signals from more than one person in one magnetic resonance imaging scanner, comprises the steps of: a) providing an MRI scanner whose dimensions and physical features of imaging components such as magnet, gradient, and RF coil can accommodate at least two people; b) such scanner can simultaneously excite the spins and simultaneously receive their magnetic resonance signals from at least two people; c) the magnetic resonance images reconstructed from such scanner can reveal either interaction and/or non-interaction between the plurality of people inside the scanner.

A method for simultaneously acquiring magnetic resonance signals from two people in one magnetic resonance imaging scanner, may also comprise the steps of: a) providing an MRI scanner which has elliptical cylinder dimension inner bore can accommodate two people; b) such scanner can simultaneously excite the spins and simultaneously receive their magnetic resonance signals from two people;

c) the magnetic resonance images reconstructed from such scanner can reveal either interaction and/or non-interaction between two people inside the scanner.

The method of may further include the steps of: a) constructing an elliptical cylinder inner bore superconducting or nonsuperconducting magnet that can generate homogeneous static magnetic field whose volume can accommodate two people; b) constructing three direction magnetic field gradient coils which can produce linear magnetic gradient region that can accommodate two people; c) constructing RF coils that can transmit and/or receive MR signals in the region that can accommodate two people; and d) constructing shimming coil set that can shim an elliptical volume that is large enough for accommodating two people.

Step b) may further include the step of acquiring MR signal for anatomic MR imaging of two people inside scanner. Step b) may further include the step of acquiring MR signal for functional MR imaging of two people inside scanner. Step b) may further include the step of acquiring MR signal for molecular MR imaging of two people inside scanner. Step b) may further include the step of acquiring MR signal for metabolic imaging of two people inside scanner.

Step c) may further include the step of reconstructing anatomic MR images of interaction and or non-interaction between two people inside scanner. Step c) may further include the step of reconstructing functional MR images of interaction and or non-interaction between two people inside scanner. Step c) may further include the step of reconstructing molecular MR images of interaction and or non-interaction between two people inside scanner. Step c) may further include the step of reconstructing metabolic MR images of interaction and or non-interaction between two people inside scanner. Step c) may further include the step of constructing two circular polarized volume coils side-by-side for two people. The two component coils are decoupled by both their geometrical orientation and decoupling interface circuit system.

The method may further include the step of analyzing the physical sexual compatibility between man and woman. The method may further include step of analyzing the psychological compatibility between man and woman.

### REFERENCES

[1] C. Frith and U. Frith, “Interacting Minds—A Biological Basis,” Science, vol. 286, pp. 1692-1695, November 1999. [0010] [2] P. R. Montague, G. Berns, J. Cohen, S. McClure, G. Pagnoni, M. Dhamala, M. Wiest, I. Karpov, R. King, N. Apple, and R. Fisher, “Hyperscanning: Simultaneous fMRI during Linked Social Interactions,” NeuroImage, vol. 16, pp. 1159-1164, 2002. [0011] [3] F. Babiloni, F. Cincotti, D. Mattia, F. de vico Fallani, A. Tocci, L. Bianchi, S. Salinari, M. Marchiani, A. Colosimo, and Astolfi, “High Resolution EEG Hyperscanning during a Card Game”, pp. 4957-4960, The 29.sup.th Annual International Conference of IEEE EMBS 2007, Lyon, France [0012] [4] R. F. Lee, R. Giaquinto, and C. Hardy, “Coupling and Decoupling Theory and its Application to the MRI Phased Array,” Magnetic Resonance in Medicine, vol. 48, pp. 203-213, 2002.

What is claimed is:

1. A method for simultaneously acquiring human brain functional magnetic resonance signals from the heads of two people in one magnetic resonance imaging scanner, the method comprising the steps of:
a. providing an MRI scanner having dimensions and physical features of imaging components including an elliptic magnet and a twin-head RF coil that can accommodate the heads of the two people;
b. such scanner being configured to simultaneously excite and receive magnetic resonance signals from the two people;
c. reconstructing magnetic resonance images from the magnetic resonance signals to reveal brain functional interaction between the people two inside the scanner.

2. The method of claim 1, wherein step a further includes the steps of:
d. constructing an elliptical cylinder inner bore superconducting magnet that can generate homogeneous static magnetic field whose volume can accommodate two people; and
e. constructing RF coils that can transmit and/or receive MR signals in the region that can accommodate the two people.

3. The method of claim 1, wherein step b further includes the step of acquiring MR signal for functional MR imaging of two people inside scanner.

4. The method of claim 2, further comprising constructing two circular polarized volume coils side-by-side for two people, the two component coils being decoupled by both their geometrical orientation and a decoupling interface circuit system.

5. The method of claim 4 further includes step of analyzing the physical sexual compatibility the two people.

6. The method of claim 4 further includes step of analyzing the psychological compatibility between the two people.

7. Apparatus for simultaneously acquiring magnetic resonance signals from two people in one magnetic resonance imaging scanner, comprising:
a. an elliptic shape of inner bore superconducting magnet that can generate homogeneous static magnetic field whose volume can accommodate more than one people;
b. an RF coil that can transmit and/or receive MR signals in the region that can accommodate the two people; and
c. an interface that can incorporate steps a and b into a standard MRI scanner to form a multiple-people MRI scanner.

8. The apparatus of claim 7, wherein component b further includes a twin-head coil comprising:
a. two conventional circular polarized volume coils side-by-side for two people;
b. the two component coils are decoupled by both their geometrical orientation and a decoupling interface circuit system.

9. The apparatus of claim 8, wherein component b further includes a scheme for decoupling circular-polarized RF coils.
